(19) United States
(12) Patent Application Publication
(10) Pub. No.: US 2009/0035286 A1
(43) Pub. Date: Feb. 5, 2009

(54) Intracoronary, intracardiac, or intravenous infusion of a mixture of autologous bone marrow derived mononuclear cells and autologous bone marrow derived mesenchymal stem cells for utilization and rescue of infarcted myocardium

(76) Inventors: Jose J. Minguell, Santiago (CL); Gabriel Perez Lasala, Covington, LA (US)

Correspondence Address: TCA CELLULAR THERAPY, LLC;ATTN: VICKI L. NICELY, 803 TRADITION DRIVE, COVINGTON, LA, 70433, US

(21) Appl. No.: 12/287,086

(22) Filed: Oct. 6, 2008

Related U.S. Application Data
(63) Continuation-in-part of application No. 11/500,317, filed on Aug. 8, 2006.

(51) Int. Cl. A61K 35/28; A61P 9/00

(52) U.S. Cl. 424/93.7

(57) ABSTRACT

The present invention is a method for improving cardiac function and myocardial regeneration in living subjects after the occurrence of myocardial infarction. The method is a combination stem cell therapy involving a mixture of bone marrow-derived mesenchymal stem cells and bone marrow derived mononuclear cells surgically implanted by using either a direct or catheter-mediated injection into damaged myocardium. Studies have shown that the implant improves heart function and myocardial regeneration and echocardiographic measurements.

### CROSS-REFERENCE TO RELATED APPLICATIONS

This application is a continuation-in-part application based on U.S. Ser. No. 11/500,317 filed on Aug. 8, 2006 entitled “Intracoronary Injection of a Mixture of Autologous Bone Marrow Derived Mononuclear Cells and Autologous Bone Marrow Derived Mesenchymal Stem Cells for Utilization and Rescue of Infarcted Myocardium.

### BACKGROUND OF THE INVENTION

### Technical Field

There are several methods to deliver cells to the heart, among them: intracoronary (by the use of a catheter), intracardiac (directly into the heart during the intraoperative procedure of coronary artery bypass grafting, CABG or by transendocardial delivery), and intravenously (direct injection into a main blood vessel in the arm, leg, etc).

Myocardial dysfunction resulting from atherosclerosis related myocardial infarction (MI) is a widespread and important cause of morbidity in the USA and mortality amongst adults. Due to scar- and ischemia-related post infarction events, clinical manifestations are enormous and heterogeneous. The damaged left ventricle undergoes progressive ‘remodeling’ and chamber dilation, with myocyte slippage and fibroblast proliferation. These events reflect an apparent lack of effective intrinsic mechanisms for myocardial repair and regeneration. Unless, deep (and still unknown) modifications are introduced in the area proximate to the damage to force proliferation of resident myocytes (Beltrami, 2001), all restorative therapies for MI must consider the use of an exogenous source of cardiomyocyte progenitors.

A main issue in the decision to be taken has been the source and nature of cells to utilize. According to preclinical studies, the choice has ranged from resident differentiated but quiescent cardiomyocytes to stem cells or cardiomyocyte progenitors (Warejcka, 1996; Wang, 2000; Siminiak, 2003). Since, a cardiac monopotential stem cell has not yet been identified, the clinical options are narrowed to the use of a multipotential stem cell exhibiting a potential to differentiate into the cardiomyocyte lineage. From this point of view, marrow-located stem cells display the required biological properties for a cell therapy approach to treat patients with myocardial infarction (Wulf, 2001; Wagers, 2002; Herzog, 2003). Using animal models, it has been reported a near-normalization of ventricular function after treatment of acute infarcted myocardium with locally-injected bone marrow-derived precursor cells (Jackson, 2001; Orlic, 2001, for a recent review, see Husnain, 2005). However, it was not clear whether the beneficial effect produced by the graft was elicited by hematopoietic stem cells, precursors for cardiomyocytes and/or endothelial cells, stem cell plasticity or just contamination with other marrow cells (Wagers, 2002). On the other hand, the transplantation of unfractionated sheep bone marrow into chronically infarcted myocardium did not result in any beneficial effect (Bel, 2003).

In addition, several studies have utilized mesenchymal stem cells (MSC) as a cell archetype for regenerative purposes after myocardial infarction. In vitro studies have shown that MSC have the potential to differentiate into spontaneous beating myotube-like structures, which express natriuretic peptides, myosin, desmin, and actinin and exhibit sinus node-like and ventricular cell-like action potentials (Makino, 1999; Bittira, 2002). In vivo studies have shown that when MSC are implanted into myocardium they undergo a milieu-dependent (microenvironment) cardiomyogenic differentiation and develop into myofibers containing striated sarcomeric myosin heavy chain and cell to cell junctions (Wang, 2000; Barbash, 2003). The xenogeneic or syngeneic transplantation of MSC have shown that infused cells were signaled and recruited to the normal and/or injured heart (Allers, 2004; Bittira, 2002), where they undergo differentiation and participate in the pathophysiology of post-infarct remodeling, angiogenesis and maturation of the scar (Bittira, 2003; Pittenger, 2005; Minguell, 2006). Furthermore, recent pig studies have shown that MSC infusion improves left ventricular function following myocardial infarction with no detectable immune or other toxicity (Min, 2002; Shake, 2002).

Thus, the results of experimental studies showing that the implant of bone marrow-derived progenitor cells improves heart function after myocardial infarction have prompted several groups to test this notion in people. In the last 3 years, various clinical studies have assessed the effect of transplantation of autologous bone marrow in myocardial regeneration after acute myocardial infarction. In all these studies, the source of “repairing” cells has been the bone marrow mononuclear cell fraction (BM-MNC), which contains B, T and NK lymphocytes, early myeloid cells, endothelial progenitors and a very low number of hematopoietic and/or mesenchymal stem cells. In these studies, bone marrow was aspirated (40-250 ml) from patients, the BM-MNC prepared and the resulting cells (10.sup.6 to 10.sup.7) implanted into the infarcted ischemic myocardium, by using either a direct or a catheter-mediated injection. Results showed that the autologous implantation procedure is safe, feasible and seems to be effective under clinical conditions (Assmus, 2002; Perin, 2003; Sekiya, 2002; Stamm, 2003; Strauer, 2002; Tse, 2003). In all cases, the observed therapeutic effect was attributed to bone marrow progenitors-associated neovascularization (angiogenesis, Rafii, 2003), thus improving perfusion of infarcted myocardium.

Based on preclinical and clinical studies, the rationale of the present clinical study is the following: every clinical attempt for myocardial regeneration might consider the implant of autologous progenitor cells, with the potential to differentiate and mature into cardiomyocytes, thus contributing to the recovery of local contractility. However, a comprehensive therapy should also consider the revascularization of the ischemic tissue by the implant of endothelial progenitor cells.

### BRIEF SUMMARY OF INVENTION

Consequently, we propose that the combined infusion of autologous purified and expanded marrow-derived mesenchymal stem cells (a source of cardiomyocyte progenitor) and autologous bone marrow mononuclear cells (a primary source of endothelial progenitors) represents an effective and enduring myocardial replacement therapy. The above presupposes that the pair of implanted autologous progenitors will express their respective biological programs after interacting with proper microenvironment locus of the receptor tissue (Minguell, 2001; Wagers, 2002; Rafii, 2003).

### DETAILED DESCRIPTION OF THE INVENTION

Results of experimental studies have shown that intramyocardial implantation of autologous mononuclear bone marrow cells induces neovascularisation, but not a robust improvement in heart function, after myocardial infarction. We propose that the above therapy in conjunction with one that provides a source of cardiomyocytes will represent a substantial promise as a cellular agent for cardiovascular therapy.

As a source of cardiomyocyte progenitors and based on in vitro, ex vivo and in vivo studies, we propose the use of autologous ex vivo expanded bone marrow-derived mesenchymal stem cells (MSC). Encouraging preliminary efficacy data in large animal models of myocardial infarction (Minguell, 2006) and accumulating safety data from human studies of MSCs in non-cardiovascular applications is encouraging.

In detail, our invention is the intracoronary injection (implant via catheter or direct injection) of a mixture of autologous bone marrow-derived mesenchymal stem cells (BM-MSCs) (cells that have the potential to differentiate and mature into mature cardiomyocytes) and autologous bone marrow-derived mononuclear cells (BM-MNCs) (cells that contain endothelial progenitors) that have the potential to differentiate and mature into cardiomyocytes and endothelial cells, representing an effective and enduring myocardial replacement therapy. See procedure below.

Primary bone marrow aspirations from the iliac crest will be performed in patients twenty-five.+−.five days before receiving the cell infusion for preparation and expansion of BM-MSC. A secondary (25.+−. 5 days from primary aspiration) bone marrow aspiration from the iliac crest for preparation of BN-MNC will be performed within 5 hours of the intracoronary cell infusion to patients. For cell infusion, aliquots of autologous expanded BM-MSC and BM-MNC are taken and mixed together for a final volume of infusion medium.

For a better understanding of procedures and schedule, please refer to the following Table.

TABLE 1
DIAGRAM OF PROCEDURES AND SCHEDULE

| Days to | | Type of sample | |
|---|---|---|---|
| infusion | Step | to be taken | Type of test to be performed |
| −25 | 1^{st} Bone marrow aspirate | cell suspension | differential cell count; |
| | for preparation of MSC | | microbiological |
| | cells | | |
| −25 | Mononuclear cell fraction | cell suspension | differential cell count |
| −20 | Passage #0 (Primary BM- | growth medium & | cell number, viability, |
| | MSC culture) | cell suspension | microbiological |
| −16 | Passage #1 | cell suspension | cell number, viability |
| −12 | Passage #2 | cell suspension | cell number, viability |
| −8 | Passage #3 | cell suspension | cell number, viability |
| −4 | Passage #4 (Expanded | growth medium & | cell number, viability, |
| | MSC) | cell suspension | microbiological, mycoplasma |
| 0 | Final preparation of BM- | BM-MSC | cell number, viability |
| | MSC | suspension | microbiological, mycoplasma, |
| | | | Gram stain, |
| | | | immunotypification, |
| | | | differentiation potential |
| 0 | 2^{nd} Bone marrow aspirate | BM-MSC | cell number, viability |
| | for preparation of MNC | suspension | microbiological, Gram stain, |
| | cells | | immunotypification |
| 0 | Cell product for infusion | BM-MSC plus | cell number, viability |
| | (final mixture of autogous | BM-MNC | microbiological, Gram stain, |
| | BM-MSC and BM-MNC) | suspension | endotoxin |

BM-MNC: bone marrow-derived mononuclear cell fraction
BM-MSC: bone marrow-derived mesenchymal stem cells

Cell infusion (transplantation) may be done in patients intraoperatively in conjunction with coronary artery bypass grafting by direct injection following the circumference of the infarct border or via intracoronary percutaneous balloon catheter designed for angioplasty. Subjects may include patients who fit criteria for acute myocardial infarction or patients with a defined region of myocardial dysfunction related to a previous myocardial infarction.

Wall motion and left ventricular ejection fraction is evaluated by MRI and echocardiography. SPECT is used to assess viability and myocardial perfusion.

A method for myocardial replacement therapy for a patient is disclosed. It involves acquiring two types of bone marrow-derived cells, a source of a therapeutically effective amount of mesenchymal stem cells that give rise to cardiomyocytes and a source of endothelial precursor cells either from mononuclear cells as such or after purification, that may give rise to new fine blood vessels. The therapeutically effective amount of mesenchymal stem cells and said mononuclear cells into an injection medium is combined. Such is injected into the patient. This method may be used wherein the step of acquiring a source of a therapeutically effective amount of mesenchymal stem cells that give rise to cardiomyocytes comprises performing a first bone marrow aspiration on said patient and producing a therapeutically effective amount of expanded bone marrow-derived mesenchymal stem cells. This method of myocardial replacement therapy may also include producing said therapeutically effective amount of autologous expanded bone marrow-derived mesenchymal stem cells, wherein the first bone marrow aspiration comprises performing said first bone marrow aspiration at least 20 days before the patient receives said injection medium, wherein said first bone marrow aspiration allows for expansion of a therapeutically effective amount of autologous expanded bone marrow-derived mesenchymal stem cells and where the performing of said first bone marrow aspiration from the patient's iliac crest.

Further, the above method for myocardial replacement therapy for the patient may include acquiring a source of a therapeutically effective amount of the autologous expanded bone marrow-derived mononuclear as a source of endothelial precursor cells and comprises performing said second bone marrow aspiration from the patient's iliac crest.

As an alternate, the method for myocardial replacement therapy for the patient of the last paragraph above may be accomplished to obtain said therapeutically effective amount of mesenchymal stem cells that give rise to cardiomyocytes and said therapeutically effective amount of endothelial precursors cells in mononuclear cells, by combining a therapeutically effective amount of aliquots of said therapeutically effective amount of autologous expanded bone marrow-derived mesenchymal stem cells and said therapeutically effective amount of endothelial precursors in mononuclear cells for a final volume of said injection medium.

As another alternate, the method for myocardial replacement therapy for the patient of the paragraphs above may be accomplished by injecting said injection medium by intraoperatively injecting said therapeutically combination of cells in injection medium comprises directly to the heart in conjunction with coronary artery bypass grafting or by any other transendocardial delivery system similar to the circumference of the infarct border.

As another alternate, the method for myocardial replacement therapy for the patient of the paragraphs above may be accomplished by injecting said injection medium by injection via intracoronary catheter.

As another alternate, the method of the paragraphs above may be accomplished by said injection medium being said therapeutically effective amount of autologous expanded bone marrow-derived mesenchymal stem cells combined with said therapeutically effective amount of endothelial precursors cells in mononuclear cells.

As another alternate, the method of the paragraphs above may be accomplished by the number of mesenchymal cells being increased in a first aspiration of bone marrow by ex vivo expansion.

As another alternate, the method of the paragraphs above may be accomplished by the second aspiration being performed only to prepare the mononuclear cells.

As another alternate, the method of the paragraphs above may be accomplished by the second aspiration occurring on the day when the amount of mesenchymal stem cells is sufficient to produce the therapeutically effective amount.

### REFERENCES

Allers C, Sierralta W D, Neubauer S, Rivera F, Minguell J J, Conget P A. Dynamic of distribution of human bone marrow-derived mesenchymal stem cells after transplantation into adult unconditioned mice. Transplantation 78, 503, 2004
Assmus B, Schachinger V, Teupe C, Britten M, Lehmann R, Dobert N, Grunwald F, Aicher A, Urbich C, Martin H, Hoelzer D, Dimmeler S, Zeiher A M. Transplantation of Progenitor Cells and Regeneration Enhancement in Acute Myocardial Infarction (TOPCARE-AMI). Circulation 2002; 06: 3009-3017.
Barbash I M, Chouraqui P, Baron J et al. Systemic delivery of bone marrow-derived mesenchymal stem cells to the infarcted myocardium. Circulation. 2003; 108: 863.
Beltrami A P, Urbanek K, Kajstura J, Yan S M, Finato N, Bussani R, Nadal-Ginard B, Silvestri F, Leri A, Beltrami C A, Anversa P. Evidence that human cardiac myocytes divide after myocardial infarction. N Engl J. Med. 2001; 344:1750-1757.
Bittira B, Kuang J Q, Al-Khaldi A, Shum-Tim D, Chiu R C. In vitro pre-programming of marrow stromal cells for myocardial regeneration. Ann Thorac Surg. 2002; 74: 1154-1159.
Bittira B, Shum-Tim D, Al-Khaldi A, Chiu R C. Mobilization and homing of bone marrow stromal cells in myocardial infarction. Eur J Cardiothorac Surg. 2003; 24: 393-398.
Herzog E L, Chai L, Krause D S. Plasticity of marrow-derived stem cells. Blood 2003; 102: 3483-3493.
Husnain H K, Ashraf M. Bone marrow stem cell transplantation for cardiac repair. Am J Physiol Heart Circ Physiol 2005; 288: H2557-H2567.
Jackson K A, Majka S M, Wang H, Pocius J, Hartley C J, Majesky M W, Entman M L, Michael L H, Hirshi K K, Godell M A. Regeneration of ischemic cardiac muscle and vascular endothelium by adult stem cells. J Clin Invest 2001; 107: 1395-1402
Makino S, Fukuda K, Miyoshi S, Konishi F, et al. Cardiomyocytes can be generated from marrow stromal cells in vitro. J Clin Invest. 1999; 103: 697-705.
Minguell J J, Erices A, Conget P. Mesenchymal stem cells. Exp. Biol. Med. 2001; 226, 507-517.
Minguell J J, Erices, A. Mesenchymal Stem Cells and the Treatment of Cardiac Disease. Experimental Biology and Medicine (in press) January issue, 2006.
Min J Y, Sullivan M F, Yang Y, Zhang J P, Converso K L, Morgan J P, Xiao Y F. Significant improvement of heart function by cotransplantation of human mesenchymal stem cells and fetal cardiomyocytes in postinfarcted pigs. Ann Thorac Surg. 2002, 74: 1568-1575.
Orlic D et al. Bone marrow cells regenerate infracted myocardium. Nature 2001; 410, 701-705.
Perin E C, Dohmann H F, Borojevic R, Silva S A, Sousa A L, et al. Transendocardial, autologous bone marrow cell transplantation for severe, chronic ischemic heart failure. Circulation. 2003; 107:2294-2302
Pittenger M F, Martin B J. Mesenchymal stem cells and their potential as cardiac therapeutics. Circ Res. 2004; 95:9-20.
Rafii S, Lyden D. Therapeutic stem and progenitor cell transplantation for organ vascularization and regeneration. Nat. Med. 2003; 9: 702-712.
Sekiya, 2002 I, Larson B L, Smith J R, Pochampally R, Cui J G, Prockop D J. Expansion of human adult stem cells from bone marrow stroma: conditions that maximize the yields of early progenitors and evaluate their quality. Stem Cells, 2002; 20: 530-541.
Shake J G, Gruber P J, Baumgartner W A, Senechal G, Meyers J, Redmond J M, Pittenger M F, Martin B J. Mesenchymal stem cell implantation in a swine myocardial infarct model: engraftment and functional effects. Ann Thorac Surg. 2002; 73: 1919-1925.
Siminiak T, Kurpisz M. Myocardial replacement therapy. Circulation 2003; 108:1167-1171
Stamm C, Westphal B, Kleine H D et al. Autologous bone-marrowtem-cell transplantation for myocardial regeneration. Lancet, 2003; 361: 45-46
Strauer B E, Brehm M, Zeus T et al. Repair of infarcted myocardium by autologous intracoronary mononuclear bone marrow cell transplantation in humans. Circulation 2002; 106: 1913-1918
Tse H F, Kwong Y L, Chan J K, Lo G, Ho C L, Lau C P. Angiogenesis in ischaemic myocardium by intramyocardial autologous bone marrow mononuclear cell implantation. Lancet. 2003; 361: 47-49.
Wagers A J, Christensen J L, Weissman I L. Cell fate determination from stem cells. Gene Therapy 2002; 9:606-612.
Wang J S, Shum-Tim D, Galipeau J, Chedrawy E, Eliopoulos N, Chiu R C. Marrow stromal cells for cellular cardiomyoplasty: feasibility and potential clinical advantages. J Thorac Cardiovasc Surg. 2000; 20: 999-1005.
Warejcka D J, Harvey R, Taylor B J, Young H E, Lucas P A. A population of cells isolated from rat heart capable of differentiating into several mesodermal phenotypes. J Surg Res 1996; 62:233-242.
Wulf G G, Jackson K A, Goodell M A. Somatic stem cell plasticity: current evidence and emerging concepts. Exp. Hematol. 2001; 29: 1361-1370

1. A method for myocardial replacement therapy for a patient comprising:
acquiring two types of bone marrow-derived cells, a source of a therapeutically effective amount of mesenchymal stem cells that give rise to cardiomyocytes and a source of endothelial precursor cells either from mononuclear cells as such or after purification, that may give rise to new fine blood vessels;
combining said therapeutically effective amount of mesenchymal stem cells and said mononuclear cells into an injection medium; and
injecting said injection medium into the patient.

2. The method for myocardial replacement therapy for the patient of claim 2, wherein acquiring a source of a therapeutically effective amount of mesenchymal stem cells that give rise to cardiomyocytes comprises performing a first bone marrow aspiration on said patient and producing a therapeutically effective amount of expanded bone marrow-derived mesenchymal stem cells.

3. The method of myocardial replacement therapy for the patient of claim 3, including producing said therapeutically effective amount of autologous expanded bone marrow-derived mesenchymal stem cells of claim 3, wherein the first bone marrow aspiration comprises:
performing said first bone marrow aspiration at least 20 days before the patient receives said injection medium, wherein said first bone marrow aspiration allows for expansion of a
therapeutically effective amount of autologous expanded bone marrow-derived mesenchymal stem cells; and
performing said first bone marrow aspiration from the patient's iliac crest.

4. The method for myocardial replacement therapy for the patient of claim 4, wherein acquiring a source of a therapeutically effective amount of the autologous expanded bone marrow-derived mononuclear as a source of endothelial precursor cells comprises:
performing said second bone marrow aspiration from the patient's iliac crest.

5. The method for myocardial replacement therapy for the patient of claim 2, wherein combining said therapeutically effective amount of mesenchymal stem cells that give rise to cardiomyocytes and said therapeutically effective amount of endothelial precursors cells in mononuclear cells, comprises combining a therapeutically effective amount of aliquots of said therapeutically effective amount of autologous expanded bone marrow-derived mesenchymal stem cells and said therapeutically effective amount of endothelial precursors in mononuclear cells for a final volume of said injection medium.

6. The method for myocardial replacement therapy for the patient of claim 2, wherein injecting said injection medium comprises intraoperatively injecting said therapeutically combination of cells in injection medium comprises directly to the heart in conjunction with coronary artery bypass grafting or by any other transendocardial delivery system similar to the circumference of the infarct border.

7. The method for myocardial replacement therapy for the patient of claim 2, wherein injecting said injection medium comprises injection via intracoronary catheter.

8. The method of claim 2, wherein said injection medium is said therapeutically effective amount of autologous expanded bone marrow-derived mesenchymal stem cells combined with said therapeutically effective amount of endothelial precursors cells in mononuclear cells.

9. The method of claim 2, wherein the number of mesenchymal cells is increased in a first aspiration of bone marrow by ex vivo expansion.

10. The method of claim 10, wherein the second aspiration is performed only to prepare the mononuclear cells.

11. The method of claim 11, wherein the second aspiration occurs on the day when the amount of mesenchymal stem cells is sufficient to produce the therapeutically effective amount.
